(19) United States
(12) Patent Application Publication
(10) Pub. No.: US 2005/0083785 A1
(43) Pub. Date: Apr. 21, 2005

(54) Heating apparatus using surface acoustic wave

(75) Inventors: Shoko Shiokawa, Yokohama (JP); Yoshikazu Matsui, Hamamatsu (JP); Jun Kondoh, Hamamatsu (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: MEDICAL & BIOLOGICAL LABORATORIES CO., LTD., Nagoya-shi (JP)

(21) Appl. No.: 10/926,336

(22) Filed: Aug. 26, 2004

(30) Foreign Application Priority Data
Sep. 16, 2003 (JP) 2003-322485
Jan. 16, 2004 (JP) 2004-009890

(51) Int. Cl. H04R 1/00

(52) U.S. Cl. 367/157; 367/140

(57) ABSTRACT

The present invention provides new applications for surface acoustic waves. A heating apparatus with a surface acoustic wave generator is disclosed. In one embodiment of the invention, a heating apparatus comprise a surface acoustic wave generator having a piezoelectric substrate and inter-digital transducers (IDT) formed on the piezoelectric substrate, and a thermal reaction part is formed at the part of the piezoelectric substrate.

### CROSS REFERENCE TO RELATED APPLICATIONS

This application claims priority to Japanese application No. 2003-322485, filed Sep. 16, 2003 and Japanese application No. 2004-009890, filed Jan. 16, 2004. The contents of the applications numbered 2003-322485 and 2004-009890 are hereby incorporated by reference in their entirety.

### FIELD OF THE INVENTION

This invention relates to apparatus using surface acoustic waves. More specifically, it relates to heating apparatus using surface acoustic waves, methods using thereof and the like.

### BACKGROUND OF THE INVENTION

The device using surface acoustic wave (also referred to as “SAW”) is widely utilized as high frequency filters for portable telephone, TV or VTR. And also, it is applied to various detectors such as the gas sensor, etc. and ultrasonic atomizer, ink jet printer, wipers, fuel injection system of the liquid (see JP 5-5869 A, JP 5-34349A, JP 7-232114A, JP 8-140898, JP 9-201961A, and JP 10-193592 A).

On the other hand, it is possible that the liquid flows, jet streams or atomizes using the SAW (see Thono, Kondoh, Matsui and Shiokawa, Proceedings of Acoustical Society of Japan 1-Q17(2002,09); Thono, Kondoh, Matsusi and Shiokawa, Technical Report of IEICE, US2002-68(2002-11)). Since such phenomenon occurs while the SAW is excited, application of it to various fields is often expected due to good responsibility. However, the research on the mechanism of this phenomenon is not sufficient.

### SUMMARY OF THE INVENTION

The purpose of the present invention is to provide new applications of the surface acoustic wave.

In order to accomplish the above purpose, the present inventors examined the relationship between atomization phenomenon and temperature from the viewpoint of the temperature arising of the SAW propagating surface. First, the piezoelectric substrate surface temperature was measured in the two conditions with or without water droplet on the substrate surface during SAW propagating. It was found that the substrate surface temperature greatly differed by the existence of the water. It means that the temperature of the water (liquid) retained on the piezoelectric substrate can be raised by the SAW excitation.

Then, the phenomenon was found that with the increase in the applied voltage, the substrate surface temperature rises. From this finding, it was considered to be able to control heating temperature by the applied voltage.

In addition, the substrate temperature did not exceed 100° C., even if applied voltage was increased to the degree in which the water retained on the piezoelectric substrate was atomized. The result shows that the SAW is effective as a heating method in case that the boiling of the object for heating must be avoided. In addition, it means that the heating by the SAW is excellent in the temperature stability.

In addition, it was found that the thermal effect by the SAW was achieved in short time. It was indicated that there was large utility value on the heating method by the SAW, when temperature rise are necessary instantaneously for the sample.

In addition, the reduced temperature after the heating stop by the SAW was steep, and the temperature was recovered in a moment to the temperature condition before the heating. From this result, it was found that rapid heating and rapid temperature-fall of the heating object became possible by using the SAW heating. Therefore, the SAW heating technique is very effective for the reaction system which repeats the change in temperature raise and fall periodically.

This invention was achieved by the above findings, and it provides the following composition.

[1] A heating apparatus comprising a surface acoustic wave generator.

[2] A heating apparatus comprising a surface acoustic wave generator having a piezoelectric substrate and inter-digital transducers (IDT) formed on the piezoelectric substrate, and a thermal reaction part is formed at the part of said piezoelectric substrate.

[3] The heating apparatus according to [2], wherein plurality of said IDT are formed in order to locate said thermal reaction part in between.

[4] The heating apparatus according to either [2]or [3], wherein said thermal reaction part consists of a concavity formed on the surface of said piezoelectric substrate.

[5] A heating apparatus comprising:
a surface acoustic wave generator having a piezoelectric substrate and inter-digital transducers (IDT) formed on the piezoelectric substrate; and
a thermal reaction vessel being composed of a material which is able to propagate surface acoustic waves and being connected directly or via another material which is able to propagate surface acoustic waves.

[6] A heating apparatus comprising:
a piezoelectric device with a piezoelectric substrate and an electrode formed on the piezoelectric substrate, which generates bulk waves; and
a substrate for propagate surface acoustic waves being connected to said piezoelectric device directly or via another material which is able to propagate surface acoustic waves, the substrate for propagate surface acoustic waves having a converter and a thermal reaction part, the converter is located in a region where the bulk waves propagate and converts the bulk waves into surface acoustic wave, the thermal reaction part is located in a region where the surface acoustic waves generated propagate.

[7] The heating apparatus according to any one of [2] to [5], further comprising a high frequency input device which controls exciting frequency, voltage and excitation time.

[8] The heating apparatus according to [7], wherein said high frequency input device applies the first exciting frequency, the first voltage and the first excitation time to said IDT when the heating apparatus is in the first state, and applies the second exciting frequency, the second voltage and the second excitation time to said IDT when the heating apparatus is in the second state.

[9] The heating apparatus according to [8], wherein the heating apparatus can become one or more states different from said first state and said second state, said high frequency input device applies in each state specific exciting frequency, specific voltage and specific excitation time to said IDT according to the state.

[10] The heating apparatus according to any one of [2] to [9], further comprising a cooler.

[11] A method of heating an object including propagating surface acoustic waves into the object.

[12] A method of heating an object including:
the first step of propagating the first surface acoustic waves with the first frequency, the first amplitude and the first excitation time into the object; and
the second step of propagating the second surface acoustic waves with the second frequency, the second amplitude and the second excitation time into the object.

[13] The method according to [12], wherein the step where surface acoustic waves are not propagate into the object is conducted between said first step and said second step.

[14] The method according to either [12] or [13], wherein one or more of the step where the surface acoustic waves with the specific frequency, the specific amplitude and the specific excitation time propagate into the object is conducted after said second step.

[15] The method according to any one of [12] to [14], wherein the set of said steps is conducted repeatedly.

This invention provides heating apparatus and heating method using the SAW as a heating means. By heating using the SAW, it becomes possible to rise in the short time the temperature of a liquid or material containing a liquid. And, the heating by the SAW is excellent in the temperature-stability, and it is easy to maintain the material at the constant temperature. In addition, the heating condition that corresponds for the purpose can be realized, because the control of the heating temperature is possible by adjusting the applied voltage.

On the other hand, it is possible that the liquid is made to vibrate or flow with the heating, because SAW has functions such as liquid vibration, jet stream or atomizing. Therefore, it is possible to promote the reaction, when for example, heating apparatus or heating method of this invention is utilized for the progress of some reactions.

### BRIEF DESCRIPTION OF THE DRAWINGS

These and other objectives and technical advantages of the present invention will be readily apparent from the following description of the preferred exemplary embodiments of the invention in conjunction with the accompanying drawings, in which:

FIG. 1 is a perspective view which shows schematically the surface acoustic wave heater 1 as one of examples of this invention.

FIG. 2 shows the composition of the high frequency power sources 40 used in the SAW heater 1.

FIG. 3 is a perspective view which shows schematically the surface acoustic wave heater 1a as another example of this invention.

FIG. 4 is a perspective view which shows schematically the surface acoustic wave heater 1b as another example of this invention.

FIG. 5 is a graph which shows measurement result of the surface temperature of the SAW device.

FIG. 6 shows the detecting area used to measure the temperature distribution in the substrate of the SAW device.

FIG. 7 is a graph which shows the measuring result of temperature distribution of propagation surface and circumference at the position as shown in FIG. 6 for the direction of perpendicular to SAW propagation direction (at 30 Vp-p).

FIG. 8 is a graph which shows the measurement result of the substrate surface temperature at duty ratio of the applied voltage to be 75%, 50%, 25% and 10%.

FIG. 9 shows schematically the composition of the surface acoustic wave generator (a) and heating phenomenon (b) due to the Rayleigh wave.

FIG. 10 shows a sectional view of the thermal reaction part formed on the piezoelectric substrate as an example. In the figure, reference numeral 110 shows piezoelectric substrate and reference numerals 111, 112 and 113 show the thermal reaction parts, respectively.

FIG. 11 is a graph which shows the temperature change for the time exciting water droplet of 10 μl from both sides IDT as a parameter of input voltage.

FIG. 12 is a graph which shows the temperature change for the time at input voltage of 15 Vp-p as a parameter of water droplet volume.

FIG. 13 is a graph which shows the relationship between the temperature and water droplet volume at 1 minute as a parameter of input voltage.

FIG. 14 is a graph which shows the temperature change for the time using glycerol aqueous solution at input voltage of 15 Vp-p as a parameter of concentration of glycerol aqueous solution.

FIG. 15 is a graph which shows the temperature at 1 minute using glycerol aqueous solution.

FIG. 16 shows a table for the relationship between viscosity and density of the glycerol aqueous solution.

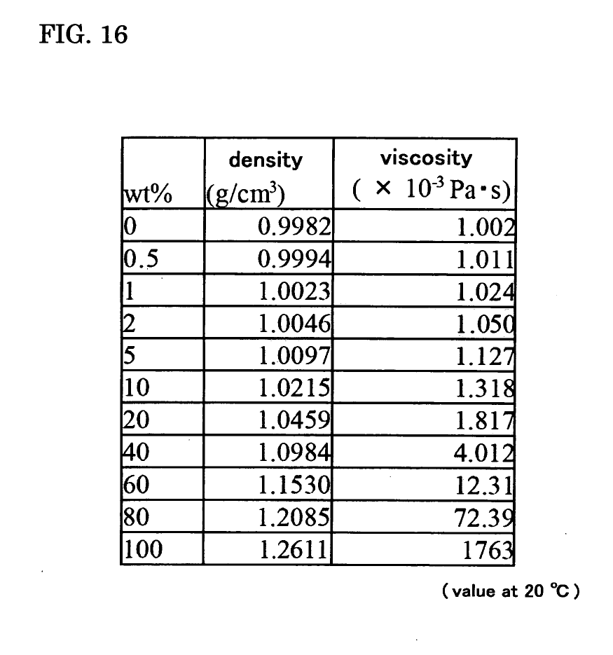

### DETAILED DESCRIPTION OF THE INVENTION

The heating apparatus of this invention has a surface acoustic wave (SAW) generator. Here, “surface acoustic wave (SAW)” is the wave which propagates along the surface of the elastic half-space in which the energy concentrated. Surface wave of (P+SV) type which contains longitudinal wave component (P wave) and transversal wave component (SV wave) with the displacement which is perpendicular to the surface is called Rayleigh wave. The research of the SAW is widely carried out in the whole world, since the IDT formed on the piezoelectric substrate would be able to realize various propagation characteristics.

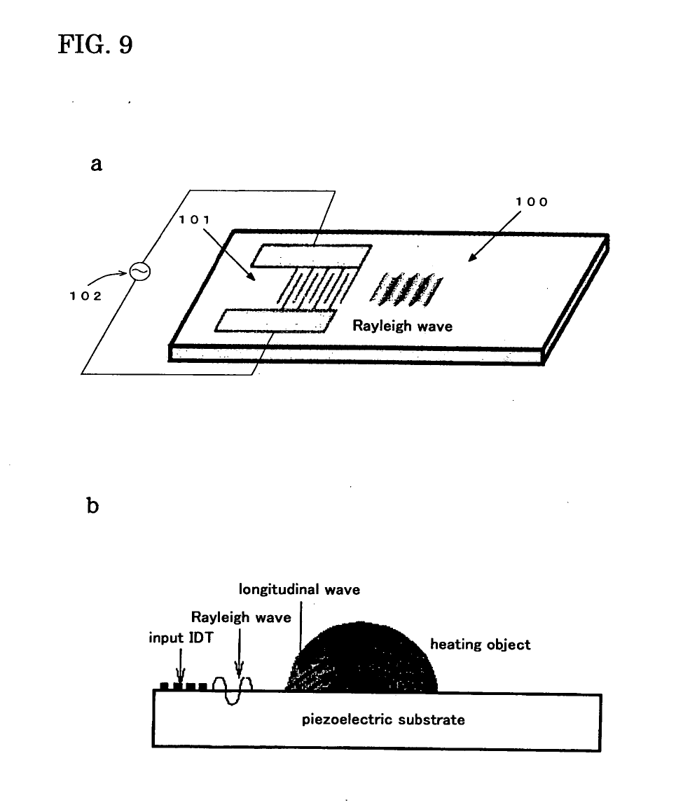

As shown in FIG. 9a, the generator of SAW generally consists of the piezoelectric substrate 100 and the IDT 101 and with which the high frequency power source 102 is electrically connected. By applying high frequency voltage to the IDT, the electric field is generated between electrodes, and the surface acoustic wave is excited, and then, it is propagated on the piezoelectric substrate. “piezoelectric” is the material which produces the strain, when the electric field is applied and when the strain is applied, the electric field is reversely produced. The materials which show the piezoelectric effect are ferroelectrics such as lithium tantalate and lithium niobate and quartz crystal and thin film of zinc oxide, etc.

In the heating apparatus of this invention, the SAW excited with the SAW generator propagates into heating object and the energy of SAW is discharged in heating object, and the energy is converted into a heat (as shown in FIG. 9b). The term “heating” in this invention means that the temperature of the object rises, and it is used as a meaning including the meaning of “warming”.

Though the heating object is not particularly limited, preferable is a liquid or a material containing a liquid. The following can be exemplified as a liquid: Water, many kind of buffer solutions, solutions for chemical reaction, culture medium used for the culture of microorganisms or cells, etc., various solutions used for process (such as etching and cleaning) of production of the semiconductor, solvents, coating material and the like. The following can be exemplified as a material containing a liquid: Cell (both of that separated from the organism and that existing in vivo are included), tissue (both of that separated from the organism and that existing in vivo are included), impregnated material with the network structure such as filter paper and polymer in the liquid or cells and organization retained in the material with the network structure,

Though the material size which can be heated by the heating apparatus of this invention is not particularly limited, it is possible to make the material of volume of several dozens of nL to several dozens of mL to be the heating object for example. Since it is possible to heat the object in short time in case the object is small, the volume of the object is preferably several dozens of nL to several hundreds of μL, more preferably several hundreds of nL to several dozens of μL.

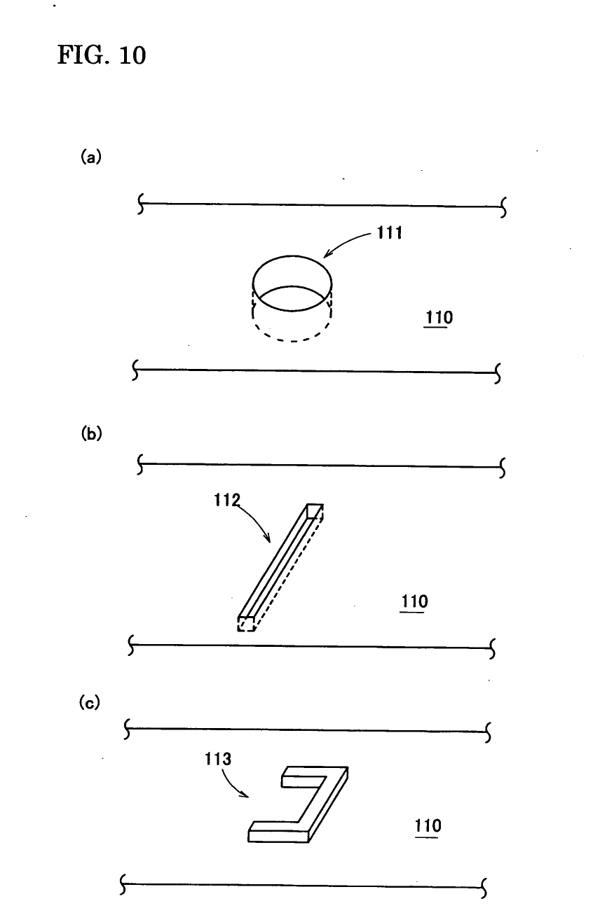

In the heating apparatus of this invention, the heating action is obtained by the SAW propagation into the heating object, so that the heating object is put in order to contact the piezoelectric substrate. In this invention, the region where the heating object contacts as above is called “thermal reaction part”. The thermal reaction part is formed in the arbitral region of the piezoelectric substrate on which SAW propagates. It is preferable that a thermal reaction part is composed of the concavity formed in the piezoelectric substrate surface in order to retain surely the heating object in the thermal reaction part. Though the concavity can be the form of pit (as shown in FIG. 10a) or groove (shown in FIG. 10b), but the shape of it is not particularly limited. And, it is also possible to make the chamber part (space), which is formed by projecting the circumference of it to form the shape of C, to be the thermal reaction part (as shown in FIG. 10c). It may be formed of plurality of thermal reaction parts. It is also possible to set the thermal reaction part of metal, paper filter or hydrophilic polymer on the piezoelectric substrate. For example, on the piezoelectric substrate, a thin film which consists of the gold is formed of the round shape on the two-dimensional plane and it is made to be the thermal reaction part on this thin film. In such thermal reaction part, the affinity for the liquid increases and as the result, the holding of the heating object improves.

It is preferable that plurality of IDTs is formed on the piezoelectric substrate so that the SAW excited by each IDT can propagate into the thermal reaction part. In such composition, the efficient heating by multiple SAW is achieved. In addition, it becomes that the heating object is located by multiple SAW, and it can be prevented that the heating object moves under heating. As a typical example, two IDT's are formed, so that the thermal reaction part is established between two IDT's. In such composition, if the SAW of the identical characteristic is excited from both side IDT's and propagate into the thermal reaction part, the heating object is efficiently heated. In addition, it become that two SAW respectively cancels flow actions of the facing SAW, the moving of the heating object can be prevented.

It is possible to arbitrary design the arrangement and shape of IDT without limiting particularly, considering relation with the thermal reaction part, intended purposes (heating object), etc. Since, it is known that the propagation width of the SAW depends on crossing width of IDT, crossing width of IDT may be designed in order to obtain SAW of propagation width which adapted to the size in the thermal reaction part.

The IDT is connected with high frequency power source with changing the applied voltage at given exciting frequency (for example, 20 MHz˜50 MHz). The heating temperature changes with the dependence on control requirement (especially, applied voltage and excitation time (pulse wave frequency and duty ratio)) of high frequency generator fed into the IDT. In other words, the heating temperature can be controlled by the condition (especially, arrangement of excitation time and applied voltage) of input high frequency. In order to enable such a regulation of high frequency, it is preferable that the heating apparatus of this invention has high frequency input means (the regulation means) for controlling operating condition of high frequency power source and inputting appropriate exciting frequency, voltage and excitation time. Using such high frequency input means, appropriate high frequency input condition which corresponds to the purpose is prepared. When the heating temperature must change, it is adjusted to high frequency input condition which corresponds to the heating temperature to be achieved. That is to say, high frequency input condition is set considering the heating temperature. For example, the high frequency input means is controlled for exciting frequency, voltage and for the excitation time so that when the heating apparatus is in the first state, the high frequency input which is under the first condition should be applied to the IDT, and when the heating apparatus is in the second state, the high frequency input which is under the second condition different from the first one should be applied to the IDT. According to such control, when the heating apparatus is in the first state, IDT can excite SAW which is adjusted for the first condition (the first SAW with the first frequency, the first amplitude and the first excitation time) and propagates into the heating object. Similarly, when the heating apparatus is in the second state, IDT can excite SAW which is adjusted for the second condition (the second SAW with the second frequency, the second amplitude and the second excitation time) and propagates into the heating object. Generally, SAW frequency is at center frequency of the frequency characteristic and SAW amplitude is the largest displacement of vibration.

In the heating using surface acoustic wave, the end-point temperature (heating temperature) is dependent on excitation characteristic of surface acoustic wave. Therefore, it becomes that two heat processing (the first and second steps) of different temperature condition is carried out by propagating two kinds of surface acoustic wave in the heating object which are generated by the control of the high frequency input as above.

The characteristics of the surface acoustic wave are decided by the excited condition (substrate, IDT pattern (number of pairs, IDT width), high frequency condition (excited frequency, voltage, excitation time (pulse frequency and duty ratio)). In other words, the surface acoustic wave with desired characteristics can be generated by configuring these conditions.

Between the above two heat steps, it may be controlled in order to carry out the step in which surface acoustic wave is not propagated in the heating object for the given time. According to such control, the temperature-fall of the heating object is quickly done after the first step. Therefore, the conversion from the first step to the second step more quickly advances, when as the first step, it heats at the high temperature, and then it heats at the lower temperature than the first step as the second process. The shortening in total heating time (reaction time) can be achieved by this process.

The heating apparatus of this invention can be composed so that it can be more than three states. In this case, it controls for frequency, voltage and for the excitation time so that the high frequency input means may be adjusted for the each condition. Though as a principle, each high frequency input condition is different from other conditions, high frequency input conditions of more than two states may be the same (as example of between two conditions or among three conditions). It is possible to set high frequency input condition of each condition considering expected thermal effect in the condition.

In above each step, the heating by peculiar surface acoustic wave is carried out for the given time in order to respectively obtain the desired heating-stage.

It becomes that the heating temperature can be controlled like the above by having adjusting mechanism for the high frequency input, and it becomes a suitable heating system (or reaction equipment) for the application in which change of heating temperature is required (for example of a series of reaction with the temperature change).

As a specific example of the application of this invention, it is possible to mention nucleic acid amplification reaction (for example, PCR (Porimerase chain reaction) method and the modification thereof (allele specific PCR and asymmetric PCR, etc.) or the method using thereof (PCR-SSCP method, etc.). In the PCR, which is a representative of Nucleic acid amplification reaction, following reactions is repeatedly done as one cycle, DNA degeneration near at 94° C., the annealing with the primer by the temperature condition of about 45° C. to about 65° C. and the polymerase elongation at near 72° C. (usually several dozens cycles). Like this, in the PCR, the temperature condition must change periodically, and the quick switching of the temperature condition is required in order to realize the efficient amplification. As it is above-mentioned, the heating apparatus of this invention can be effectively utilized for a series of reaction with periodic temperature change for such as the PCR in which it is required for quick warming and temperature-fall. And, it can adapt to various heating temperature, so that it becomes a general-purposive heating system. Properly and for example, between about 20° C. to about 90° C., heating temperature can be realized by using the heating apparatus of this invention.

In the above configuration, the thermal reaction part is formed or set in the SAW generator. The thermal reaction part can be set or provided on another member (such member which corresponds to a thermal reaction part is called “the thermal reaction vessel”) which is apart from the SAW generator. In this case, the thermal reaction vessel in which the SAW can propagate is connected directly or through other part with the piezoelectric substrate which can excite the SAW. In such composition, generated SAW propagates in the thermal reaction vessel, and then, the heating action is produced there. The material in which the SAW can propagate is, for example, piezoelectric, glass, plastics, metals, etc. The thermal reaction vessel has a region that corresponds to the thermal reaction part described above. Still, as well as the thermal reaction vessel, the above other member is constructed with the material which can propagate the SAW. However, it is well that the construction material of the thermal reaction vessel and other member is same or not.

It is also possible that the SAW which is obtained from bulk wave excited from piezoelectric device can be used as the heat source. In this case, the material for thermal reaction member (thermal reaction vessel) that can propagate SAW is connected directly or through other member with piezoelectric device used for bulk wave excitation.

The composition of the piezoelectric device which generates bulk wave is not particularly limited as long as bulk wave is generated. As the typical composition, the electrode of a metallic thin film is formed on the upper-and-lower surface of the piezoelectric substrate.

The thermal reaction vessel provides with the means for converting the bulk wave into the SAW. As a conversion means, what is called grating is used that it typically forms the stripe of metallic material layer in the part of the surface of the thermal reaction vessel or, it forms the striped groove in the part of the surface of the thermal reaction vessel.

In the case of connection of thermal reaction vessel with the piezoelectric device through other member, it may be constituted in order to convert the bulk wave at the other member into the SAW, and propagate the SAW in the thermal reaction vessel. In the case in which the surface acoustic wave is generated by the conversion of the bulk wave, the characteristic of the surface acoustic wave is dependent on the characteristic of the bulk wave, so that by establishing the methods for adjusting the characteristic of the bulk wave (the bulk wave adjusting means), the heating system with the generation of the surface acoustic wave over two types became possible. The heating apparatus with such function can heat the object under multiple thermal condition, and it becomes a suitable equipment as a heating means for a series of reaction with the temperature change required.

As above-mentioned, the SAW can eject a liquid. It is reported that as a result of this ejecting action, the size of the droplet is dependent on the frequency, and that of the droplet becomes small, as the frequency is higher (Shiokawa, Ueda and Matsui, Technical Report of IEICE, US89-51(1989) (IEICE: Inst. of Electronics, Inf. and Commun. Engineering)). Therefore, the size of the droplet decreases which occurs with the ejecting action using the heating apparatus of this invention due to the high frequency. In reverse, the size of the droplet increases, if the frequency is lowered. When the liquid is heated, it is possible to control the size of got droplet by the frequency. Especially, it becomes effective in application required to equalize the size of the droplet such as spray nozzle and ink jet nozzle or the cell sorter (cell separation device).

The SAW produces vibration, flow, flying and atomization of liquid. In the heating apparatus of this invention, it is possible to also utilize these liquid motions in addition to the heating effect. Therefore, it is also possible by controlling the applied voltage or designing equipment to vibrate simultaneously with the heating, or to flow simultaneously with the heating, or to fly or atomize simultaneously with the heating, or to vibrate and flow in addition to flying and atomization simultaneously with the heating.

Further, the heating apparatus of this invention may have a cooler. By having a cooler, the apparatus can cool the object in addition to the heating. Such composition is suitable for conducting a method or process including the multiple reactions in which the temperature condition differs. As such method, PCR (Porimerase chain reaction) method and the modification thereof (for example, allele specific PCR and asymmetric PCR) or the method using thereof (PCR-SSCP, etc.) can be exemplified.

As the cooler, the commonly used system of air-cooling and water-cooled can be applied. Concretely, the fan type cooler, the cooler using the Peltier and the like may be employed.

In the heating using the surface acoustic wave, the quick temperature-fall effect is obtained by stopping the propagation of the surface acoustic wave, as it is above-mentioned. Therefore, employing the heating apparatus of this invention, it is possible to change the temperature rise and fall for the object, even if the above described cooler is not used. However, it is preferable that the cooler is used jointly for the case in which for example, quicker cooling (temperature-fall) is necessary and case in which the sufficient cooling (temperature-fall) action is not obtained due to the large volume of the object, etc.

The heating apparatus of this invention is independently used, or it is included in other equipment. As the example of the latter case, it is included in the spray nozzle, and it is used as a heat source. For example, in etching process and cleaning treatment in the manufacturing process of the semiconductor, although the process liquid (etching and cleaning liquids) heated beforehand at the suitable temperature for each treatment is sprayed from the nozzle, the temperature of the process liquid drops, before it reaches the nozzle, and the case in which the sufficient effect is not obtained is anticipated. If the heating apparatus of this invention is included in the nozzle, the process liquid temperature in the atomization is easy to be made to be an appropriate condition because it can be heated in the nozzle.

### EXAMPLE 1

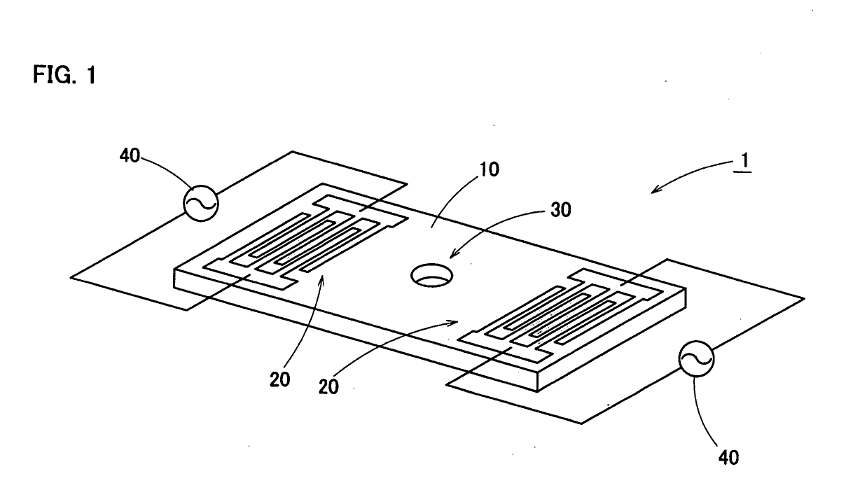

In FIG. 1, the schematics of surface acoustic wave heater (call “the SAW heater”) 1 which concerns this invention is shown. The SAW heater 1 has piezoelectric substrate 10, two inter-digital transducers 20 and thermal reaction part 30. The piezoelectric substrate 10 is made of 128 degree rotated Y cut X propagation lithium niobate (128° XY-LiNbO₃). Each inter-digital transducer 20 made of Au/Cr is formed on the surface of the piezoelectric substrate 10 in order to hold the thermal reaction part 30 from right and left. And, the distance between each inter-digital transducer 20 and thermal reaction part 30 is equal.

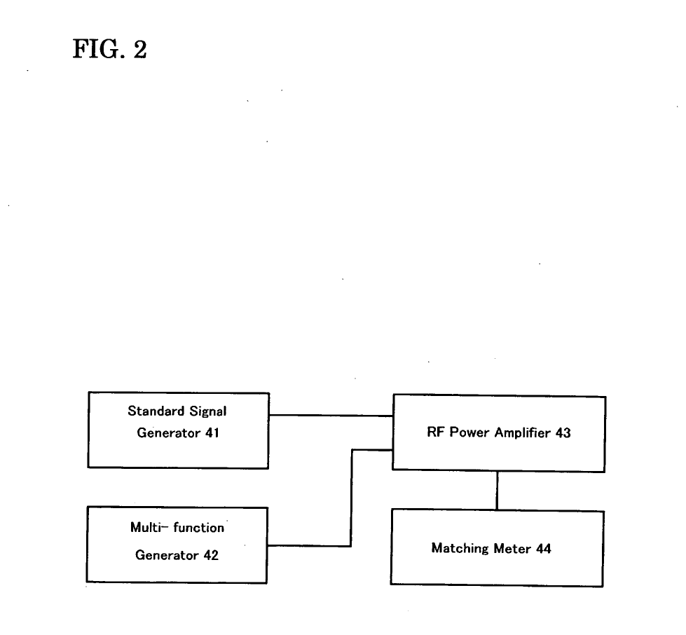

The high frequency power source 40 is connected with each inter-digital transducer 20. As shown in FIG. 2, high frequency power source 40 contains the circuit that standard signal generator 41, multi-function generator 42, RF power amplifier 43, matching meter 44 were connected. Thermal reaction division 30 consists of the cylindrical concavity formed on the surface of piezoelectric substrate 10.

Beginning of the usage of SAW heater 1, the sine wave of the fixed frequency is generated from standard signal generator 41. With this, the optional pulse signal is generated using multi-function generator 42. After two signals are mixing and amplified by using RF power amplifier 43, the signals are fed to inter-digital transducers 20 through matching meter 44. When modulated pulse signal is fed to inter-digital transducer 20, surface acoustic wave (Rayleigh wave) is excited caused by anti-piezoelectric effect. The Rayleigh wave is propagated on piezoelectric substrate 10, and arrives at thermal reaction part 30. When the liquid is retained in thermal reaction part 30, the Rayleigh wave becomes attenuated leakage Rayleigh wave though radiating the longitudinal wave into the liquid. The convection is generated in the liquid by the radiated longitudinal wave, and as the result, the temperature of the liquid rises. In SAW heater 1, the Rayleigh wave is generated by each of the inter-digital transducer 20. Therefore, the Rayleigh wave is propagated from right and left in thermal reaction part 30. As the result, it becomes a condition in which two Rayleigh waves hold the liquid located in thermal reaction part 30. By this method, it becomes possible that the liquid in thermal reaction division 30 is well retained in thermal reaction part 30. It is possible to control the heating temperature by adjusting the voltage applied to each inter-digital transducer 20.

In SAW heater 1, the material of piezoelectric substrate 10 is not particularly limited. For example, piezoelectric substrate 10 is also possible using piezoelectric ceramics. Similarly, it is possible to also use aluminum, copper, aluminum alloy, etc. except for the gold/chromium without limiting the electrode material. On the other hand, although only one is formed in this practical example as thermal reaction part 30, the multiple may be formed. And also, shape and size of the thermal reaction part 30 are not limited to it as shown in FIG. 1, respectively.

### EXAMPLE 2

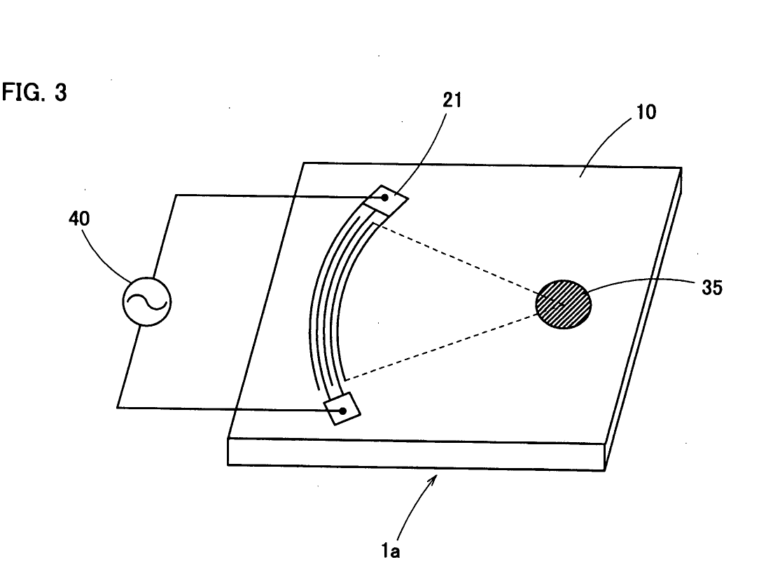

Other example is shown in FIG. 3. By appending the identical reference number to the element which is identical with that in the previous practical example and thereby its description is omitted.

In surface acoustic wave heater (the SAW heater) 1a of this example, circular arc type electrode (IDT) 21 is used, and the surface acoustic wave is converged by this, and the improvement in the heating efficiency is attempted. On piezoelectric substrate 10, the region which concentrates surface acoustic wave has been covered in a metal (for example, gold) thin film in the round shape. In SAW heater 1a, the upper surface of this metallic thin film becomes thermal reaction part 35.

### EXAMPLE 3

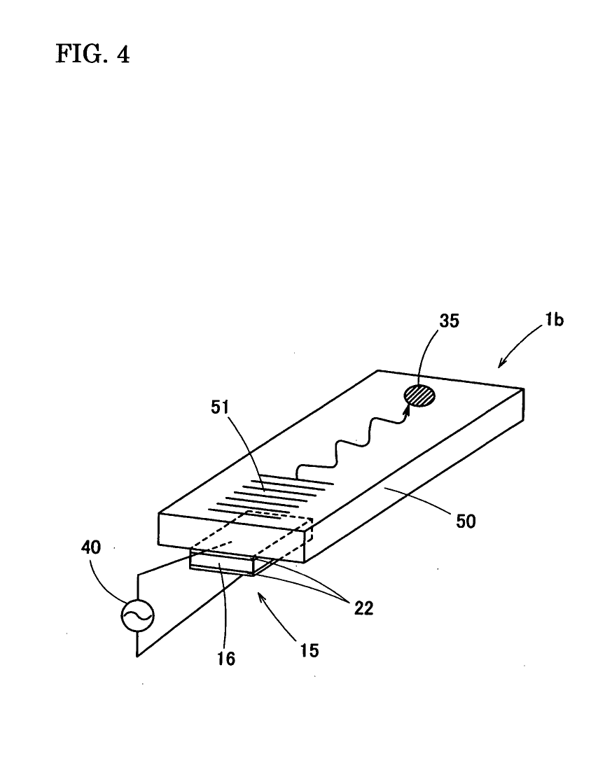

Further example is shown in FIG. 4. By appending the identical reference number to the element which is identical with that in the previous practical example and thereby its description is omitted.

For the example of the surface acoustic wave heater (SAW heater) 1b, piezoelectric devices 15, SAW propagation substrate 50 are used. Piezoelectric device 15 consists of piezoelectric substrate 16 and electrodes 22 for the bulk wave excitation. Electrode 22 for the bulk wave excitation has been formed in order to substantially cover the whole of piezoelectric substrate 16 of upper and bottom surface. In other words, electrode 22 for the bulk wave excitation is holding piezoelectric substrate 16.

SAW propagation substrate 50 is made of the glass, and it has gratings 51 made of metal layers stripe fabricated on the upper surface. In addition, thermal reaction part 35 which consists of a metallic thin film has been formed in the upper surface of SAW propagation substrate 50 in the edge region of opposite side where gratings of 51 is formed. SAW propagation substrate 50 is placed in order to contact with piezoelectric devices 15 each other as illustrated.

In SAW heater 1b, the bulk wave is generated in piezoelectric device 15 by the input of the voltage from high frequency power source. Through the contact plane with piezoelectric device of 15, this bulk wave is propagated in SAW propagation substrate 50. By the action of gratings 51, propagated bulk wave is converted into surface acoustic wave (SAW). The SAW obtained in such procedure propagates the substrate surface, and it is extensible to thermal reaction part 35.

### EXAMPLE 4

SAW device which has the same composition as one in example 1 was constructed, and the following experiments were carried out at the SAW frequency of 50 MHz. However, the thermal reaction part has not been formed on the piezoelectric substrate used for the SAW device. In the experiment, the equipment was used as follows; standard signal generator made by the leader electron Co. Ltd. No. 3220, multi-function generator made by NF circuit block Co. No. 1940, RF power amplifier made by R&K Co. Ltd. No. A100-510 and mating meter made by Daiwa Co. Ltd. No. CNW-31911.

4-1. SAW Streaming and Heating Characteristic.

Using the SAW device, the relation between heating characteristic and SAW streaming was examined. The SAW streaming is liquid motions such as flow, flying and atomizing phenomenon caused by the longitudinal wave radiation of Rayleigh wave into the liquid producing pressure difference in the liquid put on the surface of the Rayleigh wave propagation substrate.

4-1(a). The Difference in the Temperature of the Substrate Surface by the Existence of the Water

On the SAW device, the tip of the chromel-alumel thermocouple was contacted, and the surface temperature was measured. The thermocouple was contacted directly with the device surface, when there was no water. When there was the water, the thermocouple was contacted with the filter paper placed on the substrate surface in order to retain thinly the water.

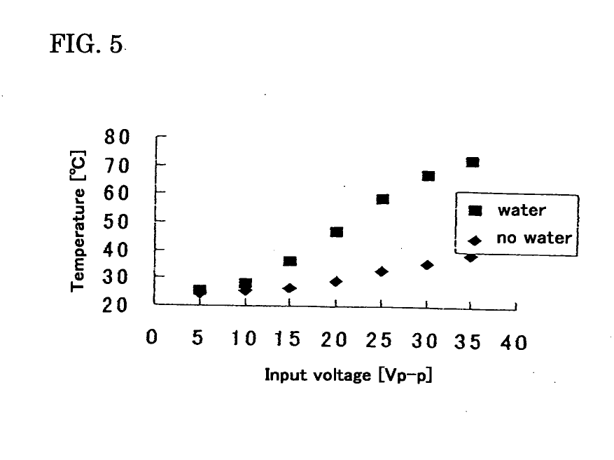

The amplitude of the input voltage to the SAW device is varied from 5V peak-to peak with 5V interval at pulse frequency of 1 kHz with Duty ratio of 50%. The measurement was carried out in the 12 second interval, after it began to apply the voltage. In this case, the water is atomized by the input voltage at 30 Vₚ₋ₚ and 35 Vₚ₋ₚ. The temperature of one point on the filter paper coming out of the fog was measured, when there was the water. The result after 1 minute is shown in FIG. 5. From the graph of FIG. 5, it is proven that the surface temperature greatly differs by the existence of the water. The temperature did not rise to 100° C., even if the applied voltage was increased to the voltage of the water atomization. From this fact, the atomization phenomenon was regarded as different with the boiling.

4-1(b) The Difference of the Temperature Distribution in SAW Propagating Surface

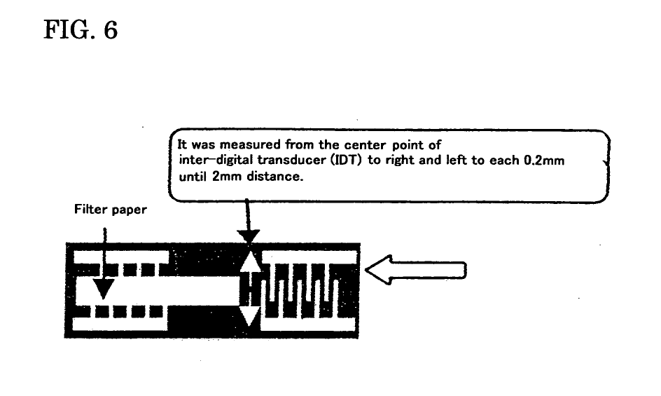

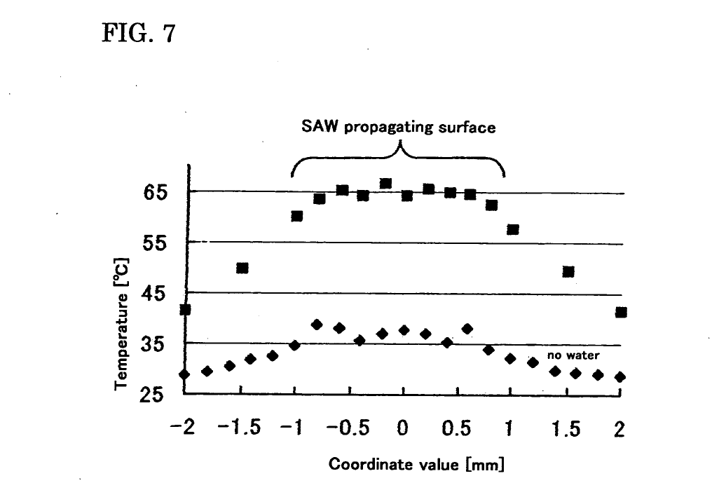

Temperature distribution in the propagating surface and its circumference were measured using the method as shown in FIG. 6 for the perpendicular direction to SAW propagation. When the water was retained on the substrate, the filter paper of No. 60 and size of 2 mm×20 mm was used in order to uniformly retain the water in propagating whole surface. The temperature after 1 minute was measured applying the input voltage of 10Vₚ₋ₚ, 20Vₚ₋ₚ and 30Vₚ₋ₚ at pulse frequency of 1 kHz. It was measured from the center point of inter-digital transducer (IDT) to right and left to each 0.2 mm until 2 mm distance. The result of 30Vₚ₋ₚ is shown in FIG. 7. From the graph of FIG. 7, it is found that when the water was retained on the substrate, the temperature in the propagating surface is the highest and almost constant. And also it is observed that the temperature in the case of water existence is very higher than in the case of without water. From this fact, it was considered that the propagation of the wave is prevented when the water is in the propagating surface, and the energy of the wave is concentrated in the water to generate large temperature rise.

4-2 The Generation of the Fog Without Accompanying of the Temperature Rise

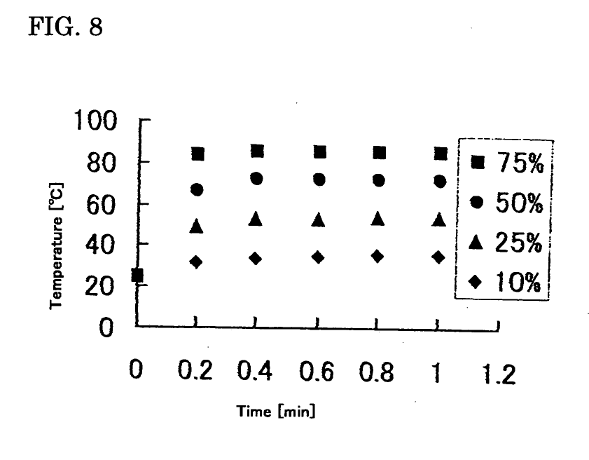

The followings were examined: The generation of the fog depends on result of the concentration of the energy or that of the amplitude of the SAW. By keeping the applied voltage of 35Vₚ₋ₚ and varying Duty ratio of 75%, 50%, 25% and 10%, the change of the substrate surface temperature was examined, and simultaneously the condition of the streaming was observed. The measurement interval was 12 seconds. The measuring result is shown in FIG. 8. From the graph of FIG. 8, it is found that the temperature is higher as the increase of the Duty ratio. From the observation of the streaming, it is found that the generation of the fog weakened as the decrease of the Duty ratio. However, the voltage at which the fog began to generation did not change, even if the Duty ratio was increased. From this fact, it is clarified that the start of the fog generation was decided by the excitation strength of the SAW.

It is shown from the above experiment that the energy of the SAW concentrates at the propagating surface by retaining the water and then the substrate surface temperature increases. It became clear that the atomization phenomenon using the SAW is based on the excitation strength of the SAW and not based on the boiling reaction. Temperature and atomization quantity could be controlled by varying the Duty ratio using the dependence of the substrate temperature and fog generation on the Duty ratio.

4-3 The Heating of the Water Droplet

The temperature was measured by putting water droplet at the center of propagating surface instead of putting the filter paper. When one electrode within the pair was excited, the temperature measurement is not possible due to the water droplet flow. Then, the SAW was excited by the pair of inter-digital transducers (IDT) designed on the substrate surface (both sides excitation). The measurement was carried out at 20Vₚ₋ₚ or less, because when the applied voltage was increased, the water moving occurs such as the flying, and since the water droplet quantity decreases.

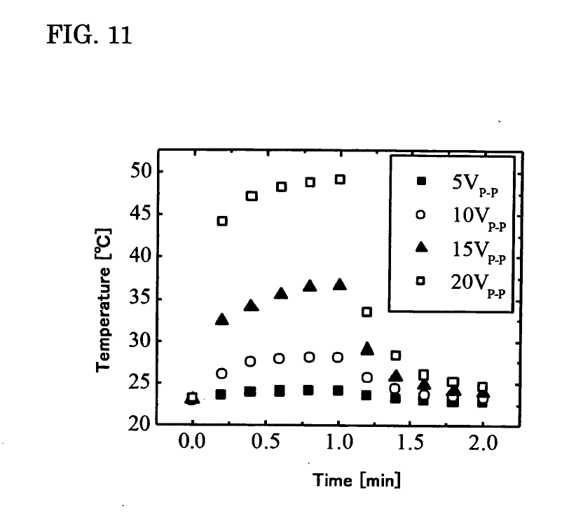

FIG. 11 is a measurement result in water droplet of 10 μl. Pf (pulse frequency) and Duty ratio were 1 kHz and 50% respectively. The input signal was applied for 1 minute. The measurement was done at the 12 second interval, and it was carried out for 2 minutes totaled in each 1 minute, exciting for 1 minute and turned off for 1 minute. As well as the case in which the filter paper was used, and it is proven that the build-up of the temperature is steep and a steady state was obtained after about 45 seconds. When the input signal is off, the temperature returns back to the initial temperature. From the result of the steep drop of the temperature in this case, it is proven that the heating is carried out only during the input signal is applied. The end-point temperature shown in FIG. 11 is higher than the case as shown in FIG. 5 in which filter paper is placed to contain the water.

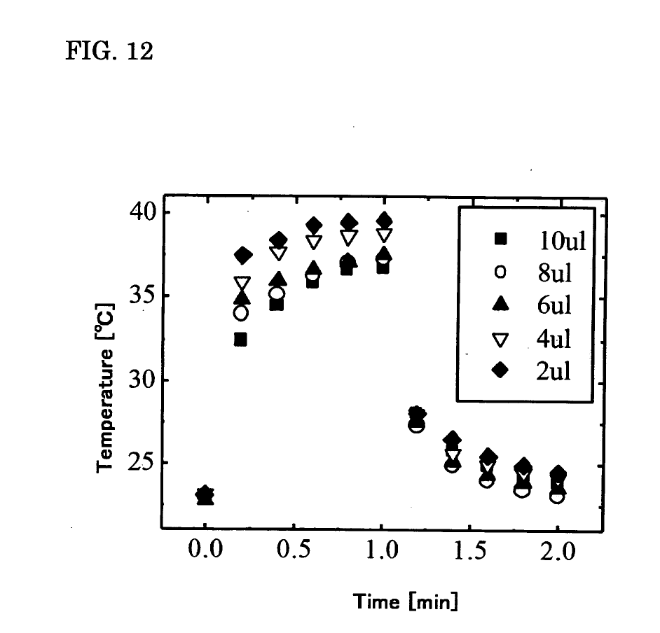

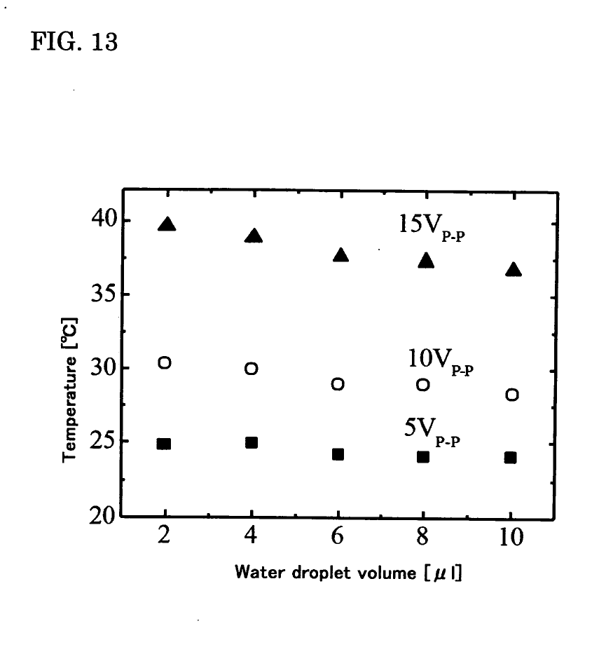

Next, the quantity of the water droplet was examined. Since stabilized measurement for small water droplet volume at 20Vₚ₋ₚ is not possible due to the water moving, the maximum applied voltage was 15Vₚ₋ₚ. The result at the applied voltage of 15Vₚ₋ₚ is shown in FIG. 12. The temperature as a function of droplet quantity after 1 minute is shown in FIG. 13 as a parameter of applied voltage from 5 Vₚ₋ₚ to 15 Vₚ₋ₚ. From these results, it is proven that the temperature increases, as the liquid volume is less. However, differences of the temperature of the water droplet for the same input voltage are the about 3 degrees. Therefore, the temperature of the droplet is strongly more dependent on the applied voltage than the quantity. FIG. 12 shows that the time at which steady state is rapid, as the volume is less. And, it is proven that the temperature of the droplet becomes more rapidly to the steady state as the volume is less. For this result the following consideration is appropriate that the thermal diffusion time of the liquid inside is shorter, as the liquid quantity is less. It is clear to be controllable for the temperature of local field from the experimental results that temperature increases while input signal is applied and when the signal is off, it decreases returning to initial temperature. This phenomenon is available for heating the part of the micro-channel as example.

4-4. The Heating of the Glycerol Aqueous Solution

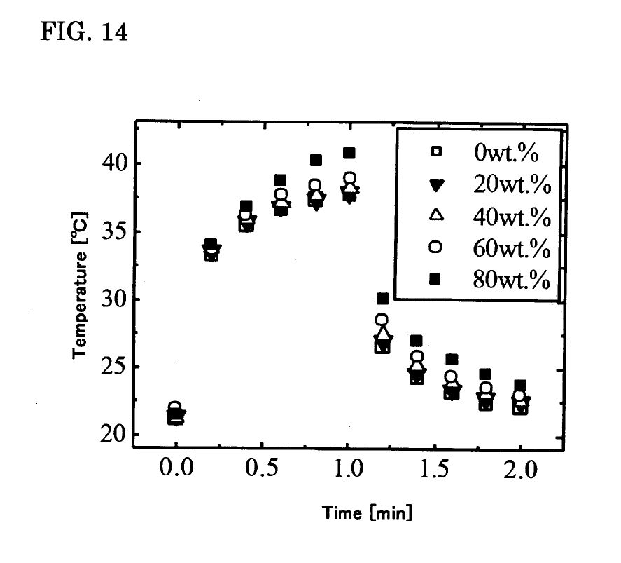

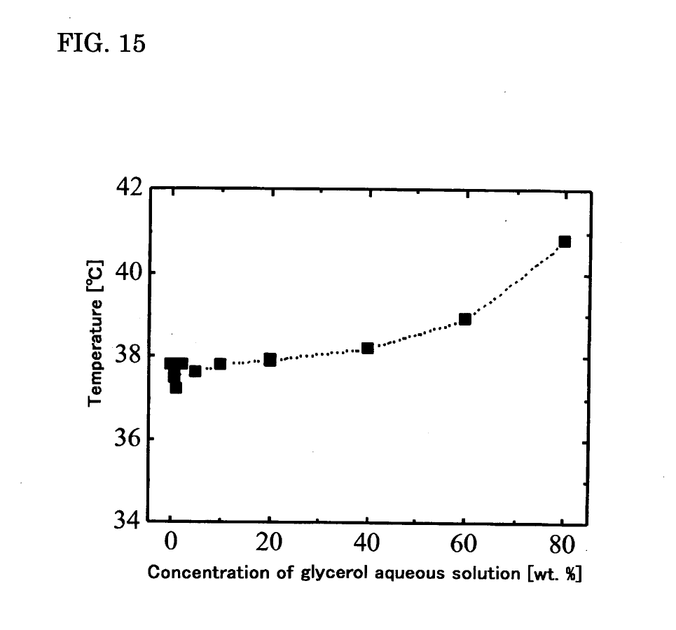

The glycerol aqueous solution was chosen as the liquid in which physical property is different from the water and the measurement was carried out by varying the glycerol concentration. In measurement, as well as shown in 4-3, the droplet was put on the center of the propagating surface using both side excitation. The applied voltage was 15Vₚ₋ₚ. FIG. 14 shows the temperature change as a function of the time. FIG. 15 shows the temperature as a parameter of the glycerol concentration at 1 minute. From these figures, it is proven that the temperature characteristic is almost equal to that of the water at the low concentration. However, it is found that at the concentration of over 40 wt. % the temperature rises further than the water. It is observed from the time response at concentration of 80 wt. % shown in FIG. 14 that the temperature does not becomes a steady state after the voltage input 1 minute. This cause is discussed based on the physical property of glycerol aqueous solution. The table of FIG. 15 shows density and viscosity for the concentration of the glycerol aqueous solution (R. C. West ed. “CRC Handbook of Chemistry and Physics, 60^{th} ed.,” CRC Press Inc., pp. D-239-D-240(1979)). From this table, it is proven that the viscosity greatly increases at the concentration over 40 wt. %. The viscous damping effect due to the radiated longitudinal wave increased at the concentration over 40 wt. %, as the result the temperature of the liquid increased.

The present invention is not limited only to the description of the above embodiments. A variety of modifications which are within the scopes of the following claims and which are achieved easily by a person skilled in the art are included in the present invention.

### Industrial Applicability

Heating apparatus or heating method of this invention can be utilized for the heating of various materials. Especially, it is suitable for heating the material of small volume. For example, it becomes the effective heating method when the heating is required in the reaction of the small sample (for example, heating and warming of the biological sample). Heating apparatus or heating method of this invention becomes the effective heating means for a series of reaction with the temperature change, because the quick temperature-fall in addition to the quick warming is possible.

The SAW produces vibration, flow, flying and atomization of liquid. In the heating apparatus of this invention, it is possible to also utilize these liquid motions in addition to the heating effect. Therefore, for example the promotion and fluidization of chemical reaction with the heating simultaneously is possible. This invention with such characteristic is utilized to various reactors and detectors, etc. This invention is also utilized as the method of spray liquid heating and warming by including this invention in spray nozzle and ink jet nozzle.

1. A heating apparatus comprising a surface acoustic wave generator.

2. A heating apparatus comprising a surface acoustic wave generator having a piezoelectric substrate and inter-digital transducers (IDT) formed on the piezoelectric substrate, and a thermal reaction part is formed at the part of said piezoelectric substrate.

3. The heating apparatus according to claim 2, wherein plurality of said IDT are formed in order to locate said thermal reaction part in between.

4. The heating apparatus according to claim 2, wherein said thermal reaction part consists of a concavity formed on the surface of said piezoelectric substrate.

5. A heating apparatus comprising:
a surface acoustic wave generator having a piezoelectric substrate and inter-digital transducers (IDT) formed on the piezoelectric substrate; and
a thermal reaction vessel being composed of a material which is able to propagate surface acoustic waves and being connected directly or via another material which is able to propagate surface acoustic waves.

6. A heating apparatus comprising:
a piezoelectric device with a piezoelectric substrate and an electrode formed on the piezoelectric substrate, which generates bulk waves; and
a substrate for propagate surface acoustic waves being connected to said piezoelectric device directly or via another material which is able to propagate surface acoustic waves, the substrate for propagate surface acoustic waves having a converter and a thermal reaction part, the converter is located in a region where the bulk waves propagate and converts the bulk waves into surface acoustic wave, the thermal reaction part is located in a region where the surface acoustic waves generated propagate.

7. The heating apparatus according to claim 2, further comprising a high frequency input device which controls exciting frequency, voltage and excitation time.

8. The heating apparatus according to claim 7, wherein said high frequency input device applies the first exciting frequency, the first voltage and the first excitation time to said IDT when the heating apparatus is in the first state, and applies the second exciting frequency, the second voltage and the second excitation time to said IDT when the heating apparatus is in the second state.

9. The heating apparatus according to claim 8, wherein the heating apparatus can become one or more states different from said first state and said second state, said high frequency input device applies in each state specific exciting frequency, specific voltage and specific excitation time to said IDT according to the state.

10. The heating apparatus according to claim 2, further comprising a cooler.

11. A method of heating an object including propagating surface acoustic waves into the object.

12. A method of heating an object including:
the first step of propagating the first surface acoustic waves with the first frequency, the first amplitude and the first excitation time into the object; and
the second step of propagating the second surface acoustic waves with the second frequency, the second amplitude and the second excitation time into the object.

13. The method according to claim 12, wherein the step where surface acoustic waves are not propagate into the object is conducted between said first step and said second step.

14. The method according to claim 12, wherein one or more of the step where the surface acoustic waves with the specific frequency, the specific amplitude and the specific excitation time propagate into the object is conducted after said second step.

15. The method according to claim 12, wherein the set of said steps is conducted repeatedly.

16. The heating apparatus according to claim 3, further comprising a high frequency input device which controls exciting frequency, voltage and excitation time.

17. The heating apparatus according to claim 4, further comprising a high frequency input device which controls exciting frequency, voltage and excitation time.

18. The heating apparatus according to claim 5, further comprising a high frequency input device which controls exciting frequency, voltage and excitation time.

19. The heating apparatus according to claim 16, wherein said high frequency input device applies the first exciting frequency, the first voltage and the first excitation time to said IDT when the heating apparatus is in the first state, and applies the second exciting frequency, the second voltage and the second excitation time to said IDT when the heating apparatus is in the second state.

20. The heating apparatus according to claim 17, wherein said high frequency input device applies the first exciting frequency, the first voltage and the first excitation time to said IDT when the heating apparatus is in the first state, and applies the second exciting frequency, the second voltage and the second excitation time to said IDT when the heating apparatus is in the second state.

21. The heating apparatus according to claim 18, wherein said high frequency input device applies the first exciting frequency, the first voltage and the first excitation time to said IDT when the heating apparatus is in the first state, and applies the second exciting frequency, the second voltage and the second excitation time to said IDT when the heating apparatus is in the second state.

22. The heating apparatus according to claim 19, wherein the heating apparatus can become one or more states different from said first state and said second state, said high frequency input device applies in each state specific exciting frequency, specific voltage and specific excitation time to said IDT according to the state.

23. The heating apparatus according to claim 20, wherein the heating apparatus can become one or more states different from said first state and said second state, said high frequency input device applies in each state specific exciting frequency, specific voltage and specific excitation time to said IDT according to the state.

24. The heating apparatus according to claim 21, wherein the heating apparatus can become one or more states different from said first state and said second state, said high frequency input device applies in each state specific exciting frequency, specific voltage and specific excitation time to said IDT according to the state.

25. The heating apparatus according to claim 3, further comprising a cooler.

26. The heating apparatus according to claim 4, further comprising a cooler.

27. The heating apparatus according to claim 5, further comprising a cooler.

28. The heating apparatus according to claim 6, further comprising a cooler.

29. The heating apparatus according to claim 7, further comprising a cooler.

30. The heating apparatus according to claim 8, further comprising a cooler.

31. The heating apparatus according to claim 9, further comprising a cooler.
